(19) United States
(12) Patent Application Publication
(10) Pub. No.: US 2025/0082957 A1
(43) Pub. Date: Mar. 13, 2025

(54) PHOTODYNAMIC THERAPY SYSTEM AND METHOD

(71) Applicants: STEBA BIOTECH S.A., Luxembourg (LU); YEDA RESEARCH AND DEVELOPMENT CO. LTD., Rehovot (IL)

(72) Inventors: Zachary Sacks, Modiin (IL); Yaniv Cohen, Ma'ale Adumim (IL); Avigdor Scherz, Tel Aviv (IL)

(21) Appl. No.: 18/726,614

(22) PCT Filed: Jan. 5, 2023
(86) PCT No.: PCT/IL2023/050019

§ 371 Date: Jul. 3, 2024

(30) Foreign Application Priority Data
Jan. 5, 2022 (IL) 289646

(51) Int. Cl. A61N 5/06

(52) U.S. Cl. CPC A61N 5/062; A61N 2005/0612; A61N 2005/0626; A61N 2005/063; A61N 2005/0632

(57) ABSTRACT

An assembly, system, and method for photodynamic therapy (PDT) of a target tissue utilizing a delivery apparatus and a displacement control arrangement. The delivery apparatus comprises a flexible needle device with an elongated flexible tube having a fiber-receiving lumen, extendable in a proximal-distal direction along a delivery axis, and with a pointed, needle-like distal tip for penetrating said tissue. An optical fiber is insertable and axially displaceable within said lumen and couplable at its proximal end to a light source, emitting a PDT-effective light at a wavelength applicable for said PDT. The fiber's distal end portion has a light-diffusing section for emitting light from within the fiber in one or more directions and has one or more X-ray markers. The displacement-control arrangement is couplable to a proximal portion of the flexible needle device and to a proximal portion of the optical fiber accommodated within the flexible needle device with the proximal portion projecting out of the proximal end of the flexible needle device, and comprises an axial displacement arrangement configured for controlled axial displacement of said delivery apparatus. The delivery apparatus may also comprise a delivery catheter extending in a proximal-to-distal direction and has a working channel that can accommodate the flexible needle device.

### TECHNICAL FIELD

The presently disclosed subject matter relates to the field of photodynamic therapy (PDT) and, more particularly, but not exclusively, to the field of vascular-targeted photodynamic therapy (VTP).

### REFERENCES

Harris K, Oakley E, Bellnier D, and Shafirstein G. J Thorac Dis. 2017, 9 (8): 2613-2618.

### BACKGROUND

Photodynamic therapy (PDT) is a form of phototherapy involving a dynamic interaction between photosensitizer, oxygen, and light to elicit cell death (phototoxicity). Photosensitizer molecules accumulate primarily in fast-growing tissues, typically cancer cells, wherein photodynamic interactions (e.g., activation of the photosensitizer molecules) are initiated by application of a light dose of a photosensitizer-specific wavelength. Once activated, the photosensitizer generates free radicals, by interaction with local oxygen, creating a highly reactive singlet oxygen form, that causes cellular damage and thereby leading to cell death or necrosis.

Vascular-targeted photodynamic therapy (VTP) is a photodynamic therapeutic technique utilizing an intravenously delivered photosensitizer that can be locally activated by light of a specific wavelength emitted by an optical fiber, typically connected to a laser. Light is applied shortly after intravenous administration of the photosensitizer, which causes a severe form of uniform local vascular injury within tissues, thereby resulting in a coagulative tissue necrosis that is tumoricidal.

To perform a minimally invasive VTP procedure, delivery of the optical fiber, and optionally additional surgical instruments, to a target tissue can be done by inserting the fiber through a catheter, which in turn can be inserted to a subject's body through their natural orifices (e.g., airways) or through a surgical incision. To reach the target tissue, while avoiding damage to the subject's body anatomy, navigation of the catheter can be performed using a navigational assist system (e.g., ultrasound-guided imaging, X-ray guidance, etc.).

References considered to be relevant as background to the presently disclosed subject matter are briefly described below. Acknowledgement of the references herein is not to be inferred as meaning that these are in any way relevant to the patentability of the presently disclosed subject matter.

Harris et al. disclose a new concept for using endobronchial ultrasound to guide interstitial PDT. For this purpose, in vitro and in vivo experiments were conducted using a phantom and animal models, respectively. A new 0.5 mm optical fiber, with a cylindrical diffuser end, was used to deliver the therapeutic light through the 21-gauge endobronchial ultrasound needle. The animal experiments were performed under real-time ultrasonography guidance in mice and rabbits' tumor models. Safe and effective fiber placements and tumor illumination were accomplished. In addition, computer simulation of light propagation suggests that locally advanced lung cancer tumors can be illuminated. This study demonstrates the potential feasibility of this new therapeutic modality approach, justifying further investigation in the treatment of locally advanced lung cancers.

### General Description

The present disclosure provides an assembly, system, and method for photodynamic therapy (PDT) of a target tissue within the body, particularly vascular-targeted photodynamic therapy (VTP).

While “PDT” is a term used in medicine to refer to photodynamic therapy in general, including topical treatment, in the context of this disclosure the term “PDT” will be used to denote such therapy intended to eradicate cells, e.g., cancerous cells, at a site within the body of a subject. The term “VTP” will be used to denote such PDT treatment intended for eradicating the vasculature within or at the vicinity of a cancerous lesion to thereby eradicate the cancer cells.

The term “target tissue” will be used herein to refer to a site of a cancer lesion, which is the target of the intended PDT or VTP. A specific example of a target tissue is a cancerous lesion within the lung.

The term “PDT-effective light” will be used herein to denote light that can activate the photosensitizing agent used in the treatment to induce its cytotoxic effect, encompassing both light for PDT or VTP.

Provided by this disclosure is an assembly for use in PDT (“PDT assembly”). Also provided by this disclosure is a system for use in PDT (“PDT system”). Further provided by this disclosure is a method for deployment of a light-diffusing section (that may also be referred to, interchangeably, as “diffuser” or “diffuser section”) at the distal end portion of an optical fiber intended for delivery of a PDT-effective light within a target tissue, as well as a PDT method. The disclosure herein enables, by certain embodiments, to deploy, under real-time X-ray guidance, the light-diffusing section of an optical fiber within a target tissue to enable delivery of PDT-effective light within said tissue.

Provided by one aspect of this disclosure is, thus, a PDT assembly that comprises a delivery apparatus and an operational displacement-control arrangement. The term “delivery apparatus” signifies a combination of elements that may be provided together in the form of a kit of discrete components that may either be separated for subsequent assembly in situ to form the delivery apparatus or already a priori assembled. The delivery apparatus may also be assembled in situ from components provided separately, e.g., each obtained from a different provider. The delivery apparatus may, by some embodiments, be a priori coupled to the displacement-control arrangement and provided as an integral kit comprising these two components. By other embodiments, the delivery apparatus and the displacement-control arrangement are independent elements that are coupled to one another prior to the PDT procedure. By some embodiments, the displacement-control arrangement is part of, or coupled to, a robotic system utilized in the performance of the PDT procedure.

The displacement-control arrangement having the functionality as described herein is another aspect of this disclosure.

The delivery apparatus of this disclosure comprises an assembly of elongated elements that extends along an axis referred to herein as a “delivery axis”. The delivery axis pertains to a line, mostly curvilinear, that may extend along a tortuous path defined by the advancement of the delivery apparatus during its deployment so that its distal end proximates the target tissue, as well as by each of its constituent elements, all of which are essentially co-axial along the same delivery axis. The delivery apparatus, and each of its constituent elements, is flexible or pliable enough to enable it to be deployed along such a tortuous path; for example, through the airways to reach a target tissue within the lung. Accordingly, the term “axis” should not be understood in a geometric sense, as meaning a straight line, but rather is used to signify a straight or tortuous line defined by the delivery apparatus, or one or more of its constituents. The axis defines an axial direction that may be tortuous and that extends along the axis; and a radial direction is normal thereto. As can be appreciated, the axial direction in one segment of the delivery catheter may not be co-axial or even parallel to that in another. Furthermore, when describing below an element or object as being able to “axially reciprocate” it should be understood as said element being able to move back and forth, in both proximal and distal directions, along the delivery axis or any segment of said axis thereof; defining said element as an axially displaceable element. As disclosed herein, the delivery apparatus comprises an elongated flexible needle device having a tubular section that extends from the proximal towards the distal end along the delivery axis, and which is configured with a pointed, needle-like tip for penetrating the target tissue; and further having a fiber-accommodating lumen for accommodating the optical fiber within (at times pre-inserted). The delivery apparatus may also comprise, by some embodiments, a delivery catheter, defining a working channel, and said flexible needle device is insertable or accommodated therein. Said flexible needle device may, by some embodiments, be part of a sheathed flexible needle device comprising a tubular sheath and a flexible needle device accommodated therein, with the optical fiber being inserted within the fiber-receiving lumen of said flexible needle device. The sheathed flexible needle device may, by some embodiments, be coupled to the displacement-control arrangement to form an integrated assembly. The delivery catheter and said flexible needle device extend in a proximal-to-distal direction defining a proximally-distally extending delivery axis.

The term “distal” refers to the end of the assembly that is inserted into the body and eventually reaches the target tissue; whereas the term “proximal” indicates the opposite end. Similarly, a “distal displacement” or “distal movement” is that which advances towards the distal end and a “proximal displacement” or “proximal movement” is that which advances in the opposite direction along the proximally-distally extending delivery axis. The terms “forward” and “rearward” may be used, interchangeably, in relation to a proximal and a distal displacement, respectively.

The term “flexible needle device” refers to an elongated flexible tube with a pointed, needle-like tip at its distal end portion.

The term “sheathed flexible needle device” refers to an assembly that comprises a flexible tubular sheath and a flexible needle device accommodated therein. The distal needle-like tip is, typically, retained within the sheath to protect the working channel and/or tissue from undesired damages that may otherwise be inflicted by the sharp tip; the flexible needle device being axially, distally displaceable to extract the tip out of the distal end of said sheathed flexible needle device for tissue penetration. A sheathed flexible needle device may be part of an assembly (“flexible needle assembly”) that comprises the sheathed flexible needle device and a manipulation element, e.g., integral, for controlled relative axial displacement of the flexible needle device and the tubular sheath, one versus the other. When used within the framework of this disclosure, the manipulation element of the flexible needle assembly may be used as part of said displacement-control arrangement.

The displacement-control arrangement may, by some embodiments, be a priori coupled to the flexible needle device or sheathed flexible needle device to form an integrated unit.

The delivery catheter is configured, by some embodiments, e.g., by means of a robotic guidance mechanism, for deployment towards the target tissue through a body lumen; for example, through the airways for reaching a target tissue within the lung, urethra for reaching a target tissue within the urinary system, etc.

The delivery catheter has a working channel for accommodating the flexible needle device or the sheathed flexible needle device. Insertable within the working channel (and optionally pre-disposed therein prior to the medical procedure in which it is used), and axially displaceable therein, is the flexible needle device (optionally, as part of a sheathed flexible needle device). As will be further elucidated by the disclosure below, the flexible needle device with its pointed distal tip is axially displaced so that said tip is extended to project out of the distal end of the PDT assembly, optionally where said PDT assembly comprises a sheathed flexible needle device, once the latter comes to be in the vicinity of the target tissue. The pointed tip projected out of said distal end permits it then to penetrate the target tissue. This manner of deployment prevents unwanted injuries to tissues other than the target tissue, which could have been inflicted otherwise had the tip been extended outside the distal end of the assembly sooner. Where said flexible needle device is part of a sheathed flexible needle device, the pointed needle-like tip is retained within the tubular sheath up to the point when it is projected out of said distal end, which ensures that the pointed tip will not damage the working channel.

The optical fiber is inserted within said fiber-receiving lumen (and typically already disposed therein prior to the medical procedure in which it is used) and axially displaceable thereof. The proximal end of the optical fiber is connectable, by an appropriate fitment, such as, for example, an SMA 905 fiber connector, to a light source emitting PDT-effective light, to permit light from said source to enter the optical fiber. According to certain embodiments, the optical fiber has a light-diffusing section at its distal end portion configured for emitting light from within the fiber in one or more directions, such as spherical, radial, radial along a section (e.g., along a predetermined irradiation length or portions thereof), radial along certain angular sectors (e.g., a 120-degree angular spread along its length), axial directions (e.g., front-face emission), and/or any clinically relevant combinations thereof. Said diffuser section comprises one or more X-ray markers, typically (albeit not exclusively) two flanking X-ray markers. The X-ray markers may, for example, be annular metallic members, e.g., made of gold or tungsten, disposed on the fiber. These X-ray markers enable real-time X-ray guidance (e.g., immediately, or substantially immediately, upon imaging one or more said X-ray markers) for proper and safe positioning of the diffuser section within the target tissue. The length of the diffuser section may be chosen such that it will span throughout the entire target tissue, for example to span along the entire length or breadth of a target tumor tissue. Alternatively, if a larger margin is desired, the diffuser may extend beyond the tumor on both or either sides, or if a smaller margin or no margin desired, the diffuser may be partially or completely within the tumor. By some embodiments, a biocompatible lubricant, such as silicon-based lubricants (e.g., Rusch® Silkospray™), may be employed that may be applied on one or both out of the optical fiber and the flexible needle device to reduce friction and facilitate smooth axial displacement of these elements.

By some embodiments, the delivery catheter has a length that is equal to or exceeds 50 cm, 60 cm, 70 cm, 100 cm, or even 140 cm.

The displacement-control arrangement may be coupled to the delivery catheter's proximal end and is configured for coupling to the proximal end of the flexible needle device, as well as to the tubular sheath (where the flexible needle device is part of a sheathed flexible needle device), and is used to control their axial displacement along both directions of the proximal-distal delivery axis as defined above. The arrangement is such to permit the combined axial displacement of at least one of the PDT assembly elements, or axial displacement of one versus the other(s). To that end, said displacement-control arrangement has a coupling arrangement permitting mechanical coupling and decoupling, of one or more of the axially displaceable assembly elements. The coupling/decoupling and displacing arrangement may be configured for joint axial displacement of two or more assembly elements or axial displacement of one versus the other(s). Joint axial displacement may be the case, for example, in extraction of the flexible needle device, optionally a sheathed flexible needle device, wherein the optical fiber is accommodated and positioned within the flexible needle device, from the distal end of the delivery catheter to approach the target tissue and penetrate it thereafter. Said arrangement also permits separate axial displacement, e.g., for retraction of the distal tip of the flexible needle device, and, optionally, the distal end of the sheathed flexible needle device, after tissue penetration, exposing the light-diffusing section at the distal end of the optical fiber and retaining it within the target tissue, as will be further elucidated below.

The coupling elements of the displacement-control arrangement can, typically, reciprocate between a rearward state and a forward state for corresponding displacement of the PDT assembly element(s) in respective proximal and distal directions. By some embodiments, one or more of these coupling elements may be independently, and selectively, decoupled from their respective assembly elements, to facilitate relative displacement of one of the assembly elements versus the other(s).

By some embodiments, the different coupling elements may be physically coupled to one another to define a reciprocating block.

In certain embodiments, at least one of the coupling elements is a Tuohy Borst adapter with a Luer connector, e.g., suitable for 2 FR diameter (although other diameters are also possible by other embodiments). In some embodiments, at least one of the coupling elements is a flapper clamp, e.g., a magnetic flapper clamp.

The displacement-control arrangement may comprise, by some embodiments, one or more displacement limiting elements defining the extent of the reciprocal displacement of the displaceable assembly elements.

The displacement-control arrangement may have several operational modes that comprise: (i) a tissue penetration mode in which a tube coupling element coupled to a proximal portion of the flexible needle device, and, optionally, to a proximal portion of the tubular sheath wherein the flexible needle device is part of a sheathed flexible needle device, and an optical fiber coupling element coupled to a proximal portion of the optical fiber are both in a coupled state for permitting joint axial displacement of said flexible needle device and said optical fiber in a distal direction and their extension thereof for penetrating the target tissue; (ii) an exposure mode in which the tube coupling element is in the coupled state and the optical fiber coupling element is in a decoupled state for retracting said flexible needle device thus exposing the light-diffusing section at the optical fiber's distal end portion and retaining it within said tissue; and (iii) an internalization mode for retracting said diffuser section back into the fiber-receiving lumen of said flexible needle device.

Provided by another aspect of this disclosure, is a PDT system that comprises a PDT assembly as described above, wherein the optical fiber is couplable at its proximal end to a light source configured to emit light at a PDT-effective wavelength. By some embodiments, the PDT system may be configured for interfacing with a robotic system for the controlled deployment of the PDT assembly; more specifically, but not limited thereto, a delivery apparatus comprising a delivery catheter accommodating therein the flexible needle device, or sheathed flexible needle device, and the optical fiber, within its working channel.

Provided by another aspect of this disclosure, is a method for deploying a light-diffusing section comprising the optical fiber's distal end portion to a subject's target tissue for performing a PDT procedure. The method comprises inserting a delivery catheter having a working channel into the subject (e.g., through the airways) and advancing it in a proximal-distal direction along the delivery axis, as described above, such that its distal end comes to be in proximity to said tissue. Then, axially displacing through the working channel a flexible needle device having a pointed, needle-like distal tip, and having a fiber-receiving lumen accommodating therein an optical fiber that is connected at its proximal end to a light source that emits light at an effective wavelength for said PDT, permitting light from said source to enter said optical fiber. The optical fiber, as already noted above, comprises a distal-end portion having a diffuser section for emitting light from within the optical fiber in a radial direction, and optionally also in an axial direction, and having one or more X-ray markers disposed therein, preferably, but not exclusively, flanking said diffuser. The distal tip portion of the flexible needle device, accommodating within its fiber-receiving lumen a distal portion of the optical fiber comprising the diffuser section, is thereby axially displaced in a proximal-distal direction, and extracted to pierce the target tissue. In some embodiments, the flexible needle device is part of a sheathed flexible needle device that is configured such that the distal pointed tip is deployable beyond the distal-end portion of the tubular sheath element of said assembly. In such cases, e.g., for deeper tissue penetration, wherein the distal tip portion of the flexible needle device is required to advance further than the maximal extraction length afforded by the delivery catheter, the entire sheathed flexible needle device can be advanced forward in a distal direction and extracted as one unit from said delivery catheter, with the needle-like tip of said flexible needle device then being extracted from the distal end of the sheath. In another embodiment, said needle-like tip can be extracted first from the sheathed flexible needle device that is accommodated within the delivery catheter, and then said sheathed flexible needle device can be extracted as well and guided into the tissue with said tip already in its extracted state.

After penetrating the target tissue, the flexible needle device may be retracted to expose the diffuser section of the optical fiber that is then retained within said tissue. The positioning of the distal end of the delivery catheter proximal to said tissue and the axial displacement of the flexible needle device, optionally, sheathed flexible needle device, having said diffuser accommodated within, is performed under real-time X-ray imaging guidance, immediately, or substantially immediately, upon imaging the one or more optical fiber X-ray markers, preferably, but not exclusively, two X-ray markers flanking said diffuser.

Provided by a further aspect of this disclosure, is a PDT method that comprises: (i) deploying a PDT assembly, of the kind described above, to bring its distal end to proximity of the target tissue, and further displacing the light-diffusing section of the optical fiber into said tissue, as also described; (ii) either before, during, or after such deployment, administering (typically intravenously) a PDT agent to the subject; (iii) activating the light source connected to said optical fiber to thereby irradiate said tissue with a PDT-effective light to be emitted from said diffuser; and (iv) removing said assembly, comprising all assembly elements, from the subject following completion of treatment.

By an embodiment of this disclosure, said PDT is VTP and said delivery catheter is deployed through a body lumen. An exemplary method of this disclosure is in the case wherein the target tissue is a cancerous lesion within the lung and the delivery catheter is deployed through the airways.

The method of this disclosure may be performed by the use of the PDT assembly and the PDT system described herein.

### EMBODIMENTS

The following are non-limiting embodiments of different aspects of the presently disclosed subject matter, intended for expanding on the above disclosure, but not to limit it in any way:

1. A PDT assembly for use in photodynamic therapy (PDT) of a target tissue, comprising:
a delivery apparatus that comprises:
   (i) a flexible needle device comprising an elongated flexible tube having a fiber-receiving lumen, extendable in a proximal-distal direction along a delivery axis, and a pointed, needle-like distal tip for penetrating said tissue; and
   (ii) an optical fiber insertable and axially displaceable within said lumen and couplable at its proximal end to a light source, emitting a PDT-effective light at a wavelength applicable for said PDT, to permit light from said source to enter the optical fiber, and said fiber having a distal end portion with a light-diffusing section for emitting light from within the fiber in one or more directions, said diffuser having one or more X-ray markers; and comprising
a displacement-control arrangement couplable to a proximal portion of the flexible needle device and to a proximal portion of the optical fiber accommodated within the flexible needle device with the proximal portion projecting out of the proximal end of the flexible needle device, and configured for selective axial displacement of said needle device and said optical fiber one versus the other.

2. A PDT assembly for use in photodynamic therapy (PDT) of a target tissue, comprising:
a delivery apparatus that comprises:
   (i) a delivery catheter having a working channel, extendable in a proximal-distal direction along a delivery axis,
   (ii) a flexible needle device comprising an elongated flexible tube having a fiber-receiving lumen and having a pointed, needle-like distal tip for penetrating said tissue, the flexible needle device being insertable and axially displaceable within said working channel; and
   (iii) an optical fiber insertable and axially displaceable within said lumen and couplable at its proximal end to a light source, emitting a PDT-effective light at a wavelength applicable for said PDT, to permit light from said source to enter the optical fiber, and said fiber having a distal end portion with a light-diffusing section for emitting light from within the fiber in one or more directions, said diffuser having one or more X-ray markers; and comprising
a displacement-control arrangement couplable to (i) a proximal portion of the flexible needle device; (ii) a proximal portion of the optical fiber accommodated within the flexible needle device with the proximal portion projecting out of the proximal end of the flexible needle device, and; (iii) a proximal portion of the delivery catheter; and comprising an axial displacement arrangement configured for controlled axial displacement of said flexible needle device, said optical fiber and said delivery catheter, and for selectively axially displacing one or more of these elements (namely one of said device, optical fiber or said catheter) versus the other(s).

3. The PDT assembly of embodiment 1 or 2, wherein said PDT is vascular-targeted photodynamic therapy (VTP).

4. The PDT assembly of any one of embodiments 1 to 3, comprising one or two X-ray markers flanking the light-diffusing section.

5. The PDT assembly of any one of embodiments 1 to 4, wherein said diffuser section is configured for emitting light in one or both of a radial and axial directions.

6. The PDT assembly of any one of embodiments 1 to 5, wherein said flexible needle device is configured for deployment through a body lumen.

7. The PDT assembly of any one of embodiments 1 to 6, wherein said optical fiber is accommodated within the fiber-receiving lumen of the flexible needle device.

8. The PDT assembly of any one of embodiments 2 to 7, wherein said flexible needle device is accommodated within said working channel, and wherein the optical fiber is accommodated within said fiber-receiving lumen.

9. The PDT assembly of any one of embodiments 1 to 8, wherein at least one of the X-ray markers is an X-ray opaque annular element.

10. The PDT assembly of any one of embodiments 1 to 9, comprising an X-ray opaque rod element at a distal end portion of the optical fiber.

11. The PDT assembly of any one of embodiments 1 to 10, wherein said flexible needle device is part of a sheathed flexible needle device that comprises a flexible tubular sheath accommodating the flexible needle device therein.

12. The PDT assembly of embodiment 11, wherein the flexible distal portion of said flexible needle device or sheathed flexible needle device is highly flexible, such that it can bend, without loss of integrity, to define a curvature with a radius of 20 mm or less.

13. The PDT assembly of any one of embodiments 11 or 12, wherein the displacement-control arrangement is configured for:
(i) retaining said distal tip within said sheath during deployment to a site proximal to said tissue; and
(ii) axially displacing the flexible needle device and extracting said distal tip following deployment.

14. The PDT assembly of embodiment 13, wherein said axial displacement is for a distance of at least 2 cm.

15. The PDT assembly of any one of embodiments 11-14, wherein the sheathed flexible needle device is part of a flexible needle assembly that comprises the sheathed flexible needle device and a manipulation element for controlled relative axial displacement of the flexible needle device and the tubular sheath, one versus the other.

16. The PDT assembly of embodiment 15, wherein the sheathed flexible needle device and the manipulation element are integral.

17. The PDT assembly of embodiments 15 or 16, wherein said manipulation element forms part of said displacement-control arrangement.

18. The PDT assembly of any one of embodiments 1 to 17, wherein the displacement-control arrangement comprises a tube coupling element that can axially reciprocate, with respect to other said displacement-control arrangement elements, between a rearward state and a forward state for respective axial displacement of said flexible needle device along the delivery axis.

19. The assembly of any one of embodiments 1 to 18, wherein the displacement-control arrangement comprises an optical fiber coupling element that can axially reciprocate, with respect to other said displacement-control arrangement elements, between a retracted state and an extended state, and having a coupled state in which it is physically coupled to said optical fiber for respective axial displacement of the fiber along the delivery axis, and further having a decoupled state in which the axial displacement of said optical fiber coupling element does not cause the axial displacement of said optical fiber.

20. The assembly of embodiment 19, wherein:
the displacement-control arrangement comprises a tube coupling element that can axially reciprocate, with respect to other said displacement-control arrangement elements, between a rearward state and a forward state, having a coupled state in which it is physically coupled to a proximal portion of said flexible needle device for respective axial displacement of the flexible needle device in both proximal and distal directions along the delivery axis, and having a decoupled state in which the axial displacement of the tube coupling element does not cause displacement of said flexible needle device; and wherein the coupling of the tube coupling element to a proximal portion of said flexible needle device and coupling of the optical fiber coupling element to said optical fiber are independent of one another permitting discrete axial displacement of the flexible needle device and the optical fiber.

21. The assembly of any one of embodiments 19 or 20, wherein said tube coupling element and said optical fiber coupling element are physically coupled to one another defining a reciprocating block.

22. The assembly of embodiment 21, wherein the displacement-control arrangement comprises displacement limiting elements defining the extent of reciprocal displacement of said reciprocating block or any of the axially displaceable elements.

23. The assembly of any one of embodiments 1 to 22, wherein the displacement-control arrangement has operational modes that comprise:
(i) a tissue penetration mode in which the flexible needle device and the optical fiber are axially displaced in a distal direction to an extended state to permit said distal tip to penetrate said tissue;
(ii) an exposure mode in which said flexible needle device is retracted in a proximal direction to expose said optical fiber's distal end portion in said tissue and retain it therein; and
(iii) an internalization mode for retracting said end portion to within said flexible needle device.

24. The assembly of embodiment 23, wherein:

in said tissue penetration mode, the tube coupling element and the optical fiber coupling element are both in their coupled state for joint axial displacement of the flexible needle device and the optical fiber in a distal direction to reach the extended state for penetration of the target tissue; and wherein
in said exposure mode, the tube coupling element is in the coupled state and the optical fiber coupling element is in the decoupled state for retracting said flexible needle device's pointed distal tip, thus exposing said optical fiber's distal end portion.

25. The assembly of any one of embodiments 18 to 24, wherein the displacement-control arrangement has operational modes that comprise:
(i) a tissue penetration mode in which the flexible needle device is axially displaced in a distal direction to the forward state to cause its pointed, needle-like distal tip to penetrate the target tissue;
(ii) a retraction mode in which said flexible needle device is axially displaced in a proximal direction to retract said distal tip from said tissue;
(iii) a fiber placement mode in which said optical fiber is axially displaced in a distal direction into a channel formed in said tissue by said distal tip; and
(iv) an internalization mode for retracting said end portion to within said flexible needle device.

26. The assembly of embodiment 25, wherein:
in said tissue penetration mode and said retraction mode the tube coupling element is in its coupled state for axial displacement of said distal tip in a distal direction to the forward state for penetrating the target tissue, and in the opposite direction, respectively; and wherein
in said fiber placement mode the optical fiber coupling element is in its coupled state for axial displacement of the optical fiber in a distal direction to the extended state for positioning and retaining said diffuser section within said tissue.

27. A PDT system for photodynamic therapy (PDT) in a target tissue, comprising an assembly of any one of embodiments 1 to 26, and a light source emitting PDT-effective light, applicable for said PDT, and being couplable to the proximal end of the optical fiber.

28. The PDT system of embodiment 27, configured for combination with a robotic system.

29. The PDT system of any one of embodiments 1 to 28 incorporated in a bronchoscope.

30. A method for deploying a light-diffusing section of an optical fiber to a target tissue in a subject for photodynamic therapy (PDT), comprising (in any applicable order of the following method elements):
axially displacing in a distal direction, a flexible needle device having an elongated tube with a pointed, needle-like distal tip and having a fiber-receiving lumen that extends in a proximal-distal direction and defines a longitudinal axis for positioning said distal tip at a site proximate to said tissue;
axially displacing in a distal direction, through said fiber-receiving lumen, an optical fiber connected at its proximal end to a light source that emits PDT-effective light to permit light from said light source to enter said optical fiber, and further having a light-diffusing section at its distal end portion for emitting said light in one or more directions, such that said diffuser section reaches said distal tip of said flexible needle device and typically remaining confined within;
axially displacing in a distal direction, said flexible needle device to cause said distal tip to pierce the target tissue and lead the distal end of the flexible needle device with the distal end portion of the optical fiber to penetrate said tissue; and
retracting said flexible needle device to expose said diffuser section and retain it within said tissue.

31. The method of embodiment 30, wherein the axial displacement of said flexible needle device is within a working channel of a delivery catheter.

32. A method for deploying a light-diffusing section of an optical fiber to a subject's target tissue for photodynamic therapy (PDT), comprising (in any applicable order of the following method elements):
axially displacing in a distal direction, a delivery catheter with a working channel that extends in a proximal-distal direction and defining a delivery axis to position the distal end of the delivery catheter proximal to said tissue;
axially displacing in a distal direction, through the working channel, a flexible needle device having a pointed, needle-like distal tip and a fiber-receiving lumen accommodating therein an optical fiber, to cause said tip to pierce the target tissue and lead the distal end of the flexible needle device with the distal end portion of the optical fiber to penetrate said tissue; said optical fiber being connected at its proximal end to a light source that emits PDT-effective light to permit light from said light source to enter said optical fiber, and further having a light-diffusing section at its distal end portion for emitting said light in one or more directions; and
retracting said flexible needle device to expose said diffuser section and retain it within said tissue.

33. A method for deploying a light-emitting segment of an optical fiber to a subject's target tissue for photodynamic therapy (PDT), comprising (in any applicable order of the following method elements):
axially displacing in a distal direction, a flexible needle device having a pointed, needle-like distal tip and a fiber-receiving lumen that extends in a proximal-distal direction and defining a delivery axis for positioning said distal tip at a site proximate to said tissue;
axially displacing in a distal direction, said flexible needle device to pierce said tissue with said distal tip and penetrate it therein;
axially displacing said flexible needle device in a proximal direction to retract said distal tip from said tissue; and
axially displacing in a distal direction through said fiber-receiving lumen an optical fiber to position the distal end portion of said optical fiber within said tissue; said optical fiber being connected at its proximal end to a light source that emits PDT-effective light to permit light from said light source to enter said optical fiber, and further having a light-diffusing section at its distal end portion for emitting said light in one or more directions.

34. The method of embodiment 33, wherein the axial displacement of said flexible needle device is within a lumen of a delivery catheter.

35. The method of any one of embodiments 30 to 34, wherein the distal end portion of said optical fiber comprises one or more X-ray markers, and wherein the axial displacement of said flexible needle device is performed under real-time X-ray imaging guidance.

36. A method for photodynamic therapy (PDT) of a target tissue of a subject, comprising (in any applicable order of the following method elements):
axially displacing in a distal direction, a flexible needle device having a pointed, needle-like distal tip and a fiber-receiving lumen that extends in a proximal-distal direction and defining a delivery axis for positioning said distal tip at a site proximate to said tissue;
axially displacing in a distal direction, through said fiber-receiving lumen, an optical fiber connected at its proximal end to a light source that emits PDT-effective light to permit light from said light source to enter said optical fiber, and further having a light-diffusing section at its distal end portion for emitting said light in one or more directions, such that said diffuser section reaches said distal tip of said flexible needle device and is confined within;
axially displacing in a distal direction, said flexible needle device to cause said distal tip to pierce the target tissue and lead the distal end of the flexible needle device with the distal end portion of the optical fiber to penetrate said tissue;
retracting said flexible needle device to expose said diffuser section and retain it within said tissue;
injecting a PDT agent to the subject;
activating said light source to thereby irradiate said tissue with said PDT-effective light emitted from said diffuser section; and
removing said flexible needle device and said optical fiber from said subject.

37. The method of embodiment 36, wherein the axial displacement of said flexible needle device is within a lumen of a delivery catheter.

38. A method for photodynamic therapy (PDT) of a target tissue of a subject, comprising (in any applicable order of the following method elements):
axially displacing in a distal direction, a delivery catheter with a working channel that extends in a proximal-distal direction and defining a delivery axis to position the distal end of the delivery catheter proximal to said tissue;
axially displacing in a distal direction, through the working channel, a flexible needle device having a pointed, needle-like distal tip and having a fiber-receiving lumen accommodating therein an optical fiber, to cause said tip to pierce the target tissue and lead the distal end of the flexible needle device with the distal end portion of the optical fiber to penetrate said tissue; said optical fiber being connected at its proximal end to a light source that emits PDT-effective light to permit light from said light source to enter said optical fiber, and further having a light-diffusing section at its distal end portion for emitting said light in one or more directions;
retracting said flexible needle device to expose said diffuser section and retain it within said tissue;
injecting a PDT agent to the subject;
activating said light source to thereby irradiate said tissue with said PDT-effective light emitted from said diffuser section; and
removing said delivery catheter with said flexible needle device and said optical fiber from said subject.

39. A method for photodynamic therapy (PDT) of a target tissue of a subject, comprising (in any applicable order of the following method elements):
axially displacing in a distal direction, a flexible needle device having a pointed, needle-like distal tip and a fiber-receiving lumen that extends in a proximal-distal direction and defining a delivery axis for positioning said distal tip at a site proximate to said tissue;
axially displacing in a distal direction, said flexible needle device to pierce said tissue with said distal tip and penetrate it therein;
axially displacing said flexible needle device in a proximal direction to retract said distal tip from said tissue;
axially displacing in a distal direction, through said fiber-receiving lumen, an optical fiber to position the distal end portion of said optical fiber within said tissue; said optical fiber being connected at its proximal end to a light source that emits PDT-effective light to permit light from said light source to enter said optical fiber, and further having a light-diffusing section at its distal end portion for emitting said light in one or more directions;
injecting a PDT agent to the subject;
activating said light source to thereby irradiate said tissue with said PDT-effective light emitted from said diffuser section; and
removing said flexible needle device with said optical fiber from said subject.

40. The method of embodiment 39, wherein the axial displacement of the flexible needle device is within a lumen of a delivery catheter.

41. A method for deploying a light-diffusing section of an optical fiber to a subject's target tissue for photodynamic therapy (PDT), comprising:
axially displacing in a distal direction, a delivery catheter with a working channel that extends in a proximal-distal direction and defining a delivery axis to position the distal end of the delivery catheter proximal to said tissue;
axially displacing in a distal direction, through the working channel, a flexible needle device having a fiber-receiving lumen and a pointed, needle-like distal tip to cause said tip to pierce the target tissue and lead the distal end of the flexible needle device to penetrate said tissue;
axially displacing said flexible needle device in a proximal direction to retract said distal tip from said tissue; and
axially displacing in a distal direction, through said fiber-receiving lumen, an optical fiber to position the distal end portion of said optical fiber within the channel formed by said distal tip in said tissue; said optical fiber being connected at its proximal end to a light source that emits PDT-effective light to permit light from said light source to enter said optical fiber, and further having a light-diffusing section at its distal end portion for emitting said light in one or more directions.

42. The method of any one of embodiments 38 to 41, wherein the distal end portion of said optical fiber comprises one or more X-ray markers; and wherein the axial displacement of said delivery catheter and said flexible needle device is performed under real-time X-ray imaging guidance.

43. The method of any one of embodiments 30 to 42, wherein said PDT is vascular-targeted photodynamic therapy (VTP); and wherein said flexible needle device is deployed through a body lumen.

44. The method of any one of embodiments 30 to 43, wherein said flexible needle device is part of a sheathed flexible needle device comprising a flexible tubular sheath accommodating said flexible needle device therein.

45. The method of embodiment 44, wherein the distal pointed, needle-like tip of said flexible needle device is accommodated within the tubular sheath and is extended out of said sheath only when positioned proximal to the target tissue.

46. The method of any one of embodiments 30 to 45, wherein the target tissue is a cancer lesion within the lung and the flexible needle device is deployed through the airways.

47. The method of embodiment 46, wherein said flexible needle device is deployed through a delivery catheter inserted through the airways.

48. The method of any one of embodiments 30 to 47, wherein the light-diffusing section of said optical fiber is flanked by two X-ray markers; and wherein said markers are used for guiding said diffuser section to its position with respect to the target tissue.

49. The method of embodiment 48, wherein at least one of the X-ray markers are X-ray opaque annular elements.

50. The method of any one of embodiments 30 to 49, comprising utilizing a system of any one of embodiments 1 to 29.

51. A displacement-control arrangement for use in the assembly of any one of embodiments 1-29 or in the method of any one of embodiments 30-50.

52. The displacement-control arrangement of embodiment 51, being couplable to a proximal portion of the flexible needle device and to a proximal portion of the optical fiber accommodated within the flexible needle device, with the proximal portion projecting out of the proximal end of the flexible needle device and configured for selective axial displacement of said flexible needle device and said optical fiber one versus the other.

53. The displacement-control arrangement of embodiment 52, being couplable to a proximal portion of the flexible needle device, to a proximal portion of the optical fiber accommodated within the flexible needle device with the proximal portion projecting out of the proximal end of the flexible needle device and couplable to a proximal portion of the delivery catheter, and comprising an axial displacement arrangement configured for controlled axial displacement of said flexible needle device, said optical fiber and said delivery catheter, and for selectively axially displacing one or more of these elements (namely one of said device, optical fiber or said catheter) versus at least one of the others.

54. The displacement control arrangement of any one of embodiments 51-53, configured for (i) retaining said distal tip within said sheath during deployment to a site proximal to said tissue; and (ii) axially displacing the flexible needle device and extracting said distal tip following deployment.

55. The displacement control arrangement of any one of embodiments 51-54, wherein
the flexible needle device is part of a sheathed flexible needle device that comprises a flexible tubular sheath accommodating the flexible needle device therein, and wherein
the displacement-control arrangement is configured for (i) retaining said distal tip within said sheath during deployment to a site proximal to said tissue, and (ii) axially displacing the flexible needle device and extracting said distal tip following deployment.

56. The displacement control arrangement of embodiment 55, wherein
the sheathed flexible needle device is part of a flexible needle assembly that comprises the sheathed flexible needle device and a manipulation element for controlled relative axial displacement of the flexible needle device and the tubular sheath, one versus the other, and
wherein said manipulation element forms part of said displacement-control arrangement.

57. The displacement control arrangement of embodiment 56, wherein the manipulation element is integral with the sheathed flexible needle device.

58. The displacement control arrangement of any one of embodiments 51-57, comprising a tube coupling element that can axially reciprocate, with respect to other said displacement-control arrangement elements, between a rearward state and a forward state for respective axial displacement of said flexible needle device along the delivery axis.

59. The displacement control arrangement of any one of embodiments 51-58, comprising an optical fiber coupling element that can axially reciprocate, with respect to other said displacement-control arrangement elements, between a retracted state and an extended state, and having a coupled state in which it is physically coupled to said optical fiber for respective axial displacement of the fiber along the delivery axis, and further having a decoupled state in which the axial displacement of said optical fiber coupling element does not cause the axial displacement of said optical fiber.

60. The displacement control arrangement of any one of embodiments 51-59, comprising a tube coupling element that can axially reciprocate, with respect to other said control device elements, between a rearward state and a forward state, having a coupled state in which it is physically coupled to a proximal portion of said flexible needle device for respective axial displacement of the flexible needle device in both proximal and distal directions along the delivery axis, and having a decoupled state in which the axial displacement of the tube coupling element does not cause displacement of said flexible needle device; the coupling of the tube coupling element to a proximal portion of said flexible needle device and coupling of the optical fiber coupling element to said optical fiber are independent of one another permitting discrete axial displacement of the flexible needle device and the optical fiber.

61. The displacement control arrangement of any one of embodiments 58-60, wherein said tube coupling element and said optical fiber coupling element are physically coupled to one another defining a reciprocating block.

62. The displacement control arrangement of any one of embodiments 51-61, having operational modes that comprise
(i) a tissue penetration mode in which the flexible needle device and the optical fiber are axially displaced in a distal direction to an extended state to permit said distal tip to penetrate said tissue;
(ii) an exposure mode in which said flexible needle device is retracted in a proximal direction to expose said optical fiber's distal end portion in said tissue and retain it therein; and
(iii) an internalization mode for retracting said end portion to within said flexible needle device.

63. The displacement control arrangement of embodiment 62, wherein
in said tissue penetration mode, the tube coupling element and the optical fiber coupling element are both in their coupled state for joint axial displacement of the flexible needle device and the optical fiber in a distal direction to reach the extended state for penetration of the target tissue; and wherein
in said exposure mode, the tube coupling element is in the coupled state and the optical fiber coupling element is in the decoupled state for retracting said flexible needle device's pointed distal tip, thus exposing said optical fiber's distal end portion.

64. The displacement control arrangement of any one of embodiments 51-63, having operational modes that comprise:
(i) a tissue penetration mode in which the flexible needle device is axially displaced in a distal direction to the forward state to cause its pointed, needle-like distal tip to penetrate the target tissue;
(ii) a retraction mode in which said flexible needle device is axially displaced in a proximal direction to retract said distal tip from said tissue;
(iii) a fiber placement mode in which said optical fiber is axially displaced in a distal direction into a channel formed in said tissue by said distal tip; and
(iv) an internalization mode for retracting said end portion to within said flexible needle device.

65. The displacement control arrangement of embodiment 64, wherein:
in said tissue penetration mode and said retraction mode the tube coupling element is in its coupled state for axial displacement of said distal tip in a distal direction to the forward state for penetrating the target tissue, and in the opposite direction, respectively; and wherein
in said fiber placement mode the optical fiber coupling element is in its coupled state for axial displacement of the optical fiber in a distal direction to the extended state for positioning and retaining said diffuser section within said tissue.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1C are general schematic representations of an exemplary system for use in photodynamic therapy (PDT) of a target tissue, namely a vascular-targeted therapy (VTP) of a cancer lesion within the lung, wherein: FIG. 1A is an overall, general schematic view of the system; FIG. 1B is a schematic magnified view on a distal portion of the delivery apparatus as it is deployed and positioned within the body; and FIG. 1C provides an additional schematic of the system, including a magnified view of the light-diffusing section, during light irradiation of the target tissue within the body of a subject.

FIG. 2 is a schematic longitudinal cross-section through the displacement-control arrangement of the system of FIGS. 1A-1C.

FIG. 3A is a schematic representation of a PDT system, according to another embodiment of this disclosure, having a different engagement mechanism with the elements of the delivery apparatus, chiefly the flexible needle device.

FIG. 3B is a magnified view of the displacement-control arrangement of the system of FIG. 3A.

FIG. 4 is a schematic longitudinal cross-section through a PDT assembly, by one embodiment, showing four operational stages of a treatment mode represented in the displacement-control arrangement; as well as the mechanistic outcome of each operational stage at the distal end portion of the PDT assembly while deployed in the target tissue.

FIG. 5 is a schematic longitudinal cross-section through a PDT assembly, by another embodiment, showing five operational stages of a treatment mode represented in the displacement-control arrangement; as well as the mechanistic outcome of each operational stage at the distal end portion of the PDT assembly while deployed in the target tissue.

FIG. 6 illustrates a generalized flowchart of a sequence of operations carried out for PDT at a target tissue of a subject, in accordance with certain embodiments of the presently disclosed subject matter.

### DETAILED DESCRIPTION

In the following detailed description, numerous specific details are set forth in order to provide a thorough understanding of the presently disclosed subject matter. However, it will be understood by those skilled in the art that the presently disclosed subject matter may be practiced without these specific details. In other instances, well-known methods, procedures, and components have not been described in detail so as not to obscure the presently disclosed subject matter.

In the figures and descriptions set forth, identical reference numerals indicate those components that are common to different embodiments or configurations.

Further, it will be appreciated that for simplicity and clarity of illustration, the figures are schematic, and elements shown in the figures have not necessarily been drawn to scale. For example, the dimensions of some of the elements may be exaggerated relative to other elements for clarity.

As used herein, the phrase “for example,” “such as”, “for instance”, and variants thereof, describe non-limiting embodiments of the presently disclosed subject matter. Reference in the specification to “one case”, “some cases”, “other cases”, or variants thereof, means that a particular feature, structure or characteristic described in connection with the embodiment(s) is included in at least one embodiment of the presently disclosed subject matter. Thus, the appearance of the phrase “one case”, “some cases”, “other cases”, or variants thereof, does not necessarily refer to the same embodiment(s).

Also, directions or orientations may be described with reference to the manner in which the elements are illustrated in the drawings. Thus, for example, the terms “above”, “below”, etc. may be used for convenience in reference to the drawings; it being understood that in real-life use the relative orientation may be different such that two elements that are one above the other may be side-by-side, etc.

It is appreciated that, unless specifically stated otherwise, certain features of the presently disclosed subject matter, which are, for clarity, described in the context of separate embodiments, may also be provided in combination in a single embodiment. Conversely, various features of the presently disclosed subject matter, which are, for brevity, described in the context of a single embodiment, may also be provided separately or in any suitable sub-combination.

According to certain embodiments of the presently disclosed subject matter, there is provided an assembly configured for use in photodynamic therapy (PDT) of a target tissue. By way of non-limiting example, the PDT may be a vascular targeted photodynamic therapy (VTP) making use of PDT agents, such as Padeliporfin (WST11). An exemplary embodiment, as also illustrated in several of the figures and described in the corresponding description below, relates to the treatment of lung cancer in which the delivery apparatus is deployed through the airways until the distal end comes into proximity of a tumor, whereupon the sequence of operations described below is executed. It is understood that this is but an exemplary embodiment of the entire scope of the present disclosure.

The exemplified embodiments described below are of a system that comprises a delivery catheter. This should not be construed as limiting, and as noted in the general description above, the teaching of this disclosure may also be embodied in a system that does not include a delivery catheter. Furthermore, attention is provided to specific embodiments that may comprise a sheathed flexible needle device, wherein the flexible needle device is sheathed within a tubular sheath. It should not be understood by the description of this specific embodiment that the presently disclosed subject matter is limited to such use, but rather also encompasses such instances wherein the flexible needle device is configured for use without such sheath.

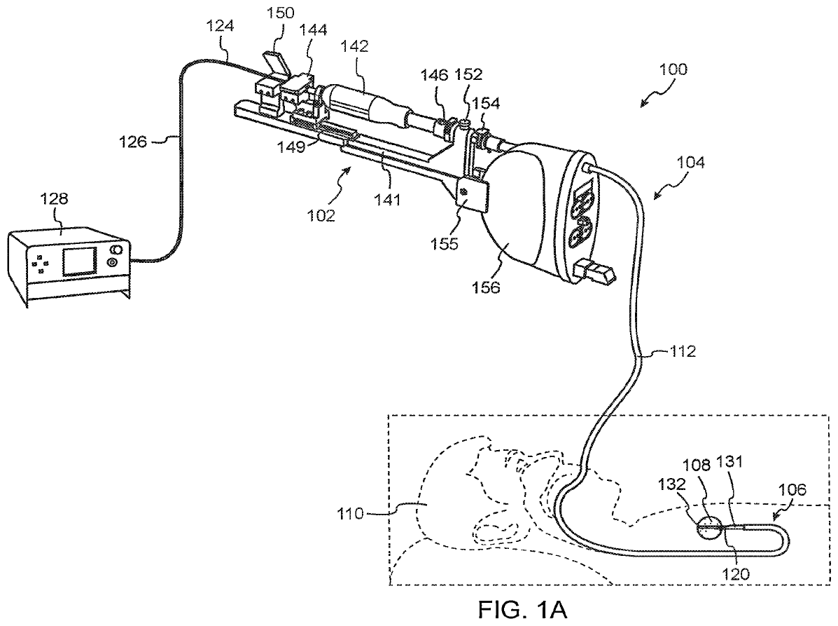

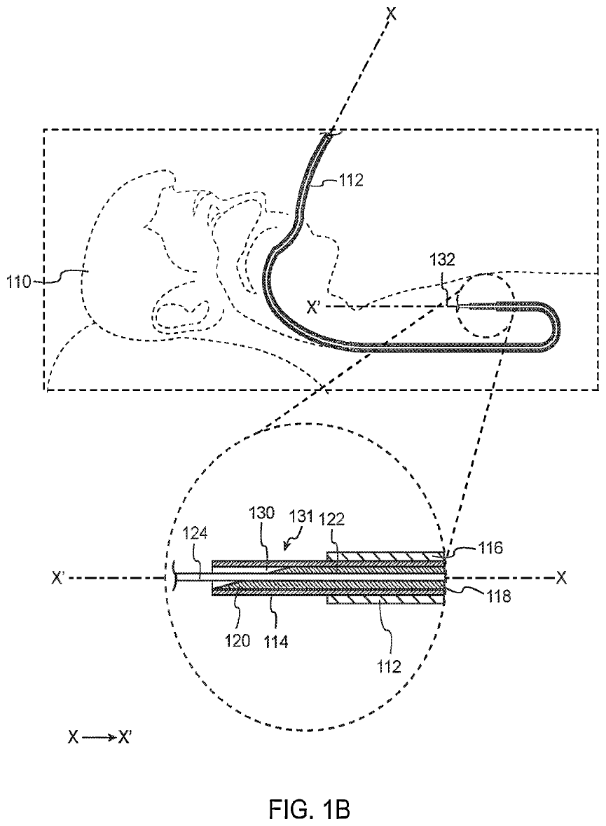

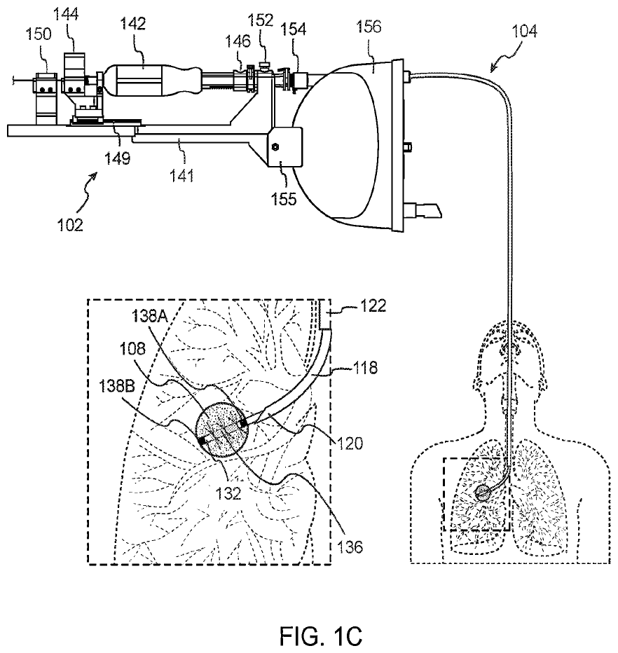

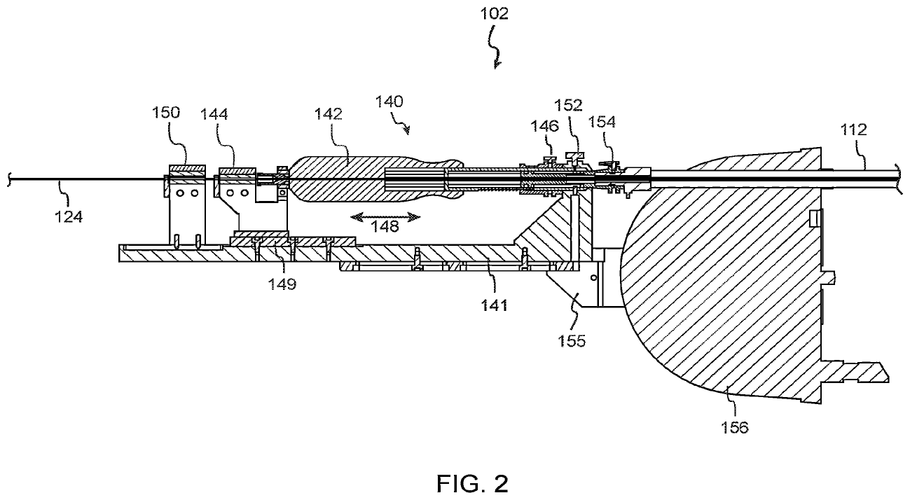

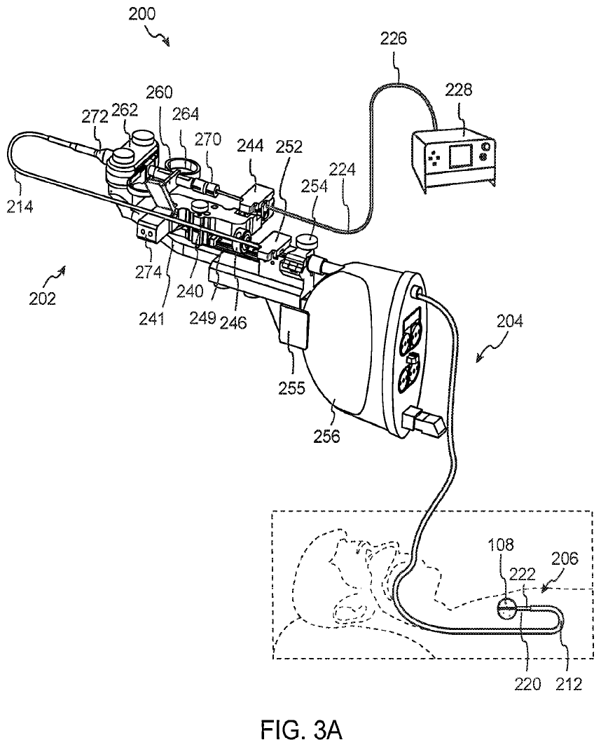

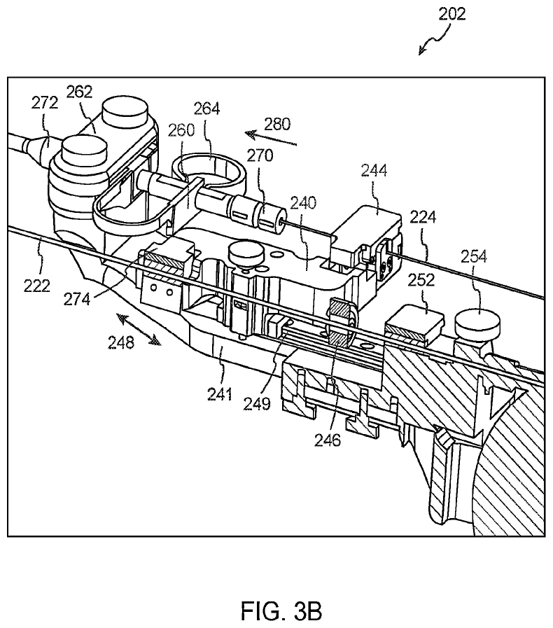

Of the embodiments specifically described below, that of FIGS. 1A-2 shows a specific embodiment using an exemplary flexible needle that only allows for the distal needle-like tip portion of the sheathed flexible needle device to be inserted into the tissue using a single step penetration process common for biopsy needles. FIGS. 3A-3B show another specific embodiment using an exemplary flexible needle assembly that allows for insertion of both the needle-like tip and sheath into the tissue by a two-step penetration process, wherein the needle may be first extended and then distal portions of the delivery apparatus may be displaced forward, while the needle-like tip is extended, to cover more distance allowing for deeper tissue penetration.

Bearing this in mind, attention is now drawn to schematic illustrations of an exemplary system shown in FIGS. 1A-1C. A PDT system 100, in accordance with the presently disclosed subject matter, comprises a displacement-control arrangement 102 coupled to a delivery apparatus 104 that extends in a proximal-to-distal direction from the displacement-control arrangement 102, with its distal end portion 106 being deployed in proximity to the target tissue 108 within the subject 110. In this exemplary embodiment, the delivery apparatus 104 is deployed and extends through the airways of subject 110 for PDT-based ablation of the target tissue 108, which in this example may be a cancerous lesion within the lung. It should, however, be noted that the delivery apparatus 104 may also be deployed through other tubular organs, or may be deployed through an incision through the skin, etc.

The delivery apparatus 104 is of an overall elongated, flexible structure and extends along and defines a tortuous delivery axis X that is schematically depicted in FIG. 1B, wherein X′ signifies a relative distal end for each particular segment along delivery axis X. The constituting components of the delivery apparatus 104 can best be appreciated in the magnified section of FIG. 1B. It includes a delivery catheter 112, a sheathed flexible needle device 114, accommodated within the catheter's working channel 116. The sheathed flexible needle device 114 comprises a flexible needle device 118 that accommodates the optical fiber 124 within its fiber-receiving lumen 130. The flexible needle device 118 has an elongated tube portion (not depicted in these figures) that ends with a pointed, needle-like distal tip 120 and is sheathed by a tubular sheath 122. The distal tip 120 is configured, once extracted out of sheath 122, for penetrating the target tissue. The main function of sheath 122 is to protect the working channel 116 of the delivery catheter 112, as well as tissues other than the target tissue, from damage that may occur during its deployment through the working channel 116. As can be understood, for the purpose of tissue penetration, sheath 122 may be retracted with respect to the flexible needle device 118, or the latter may be advanced with respect to the former thereby exposing the distal tip 120 that may then be axially advanced to penetrate the target tissue 108.

An optical fiber 124, the proximal end 126 of which is connected to a light source 128, typically, albeit not exclusively, a laser, configured to emit light at a PDT-effective wavelength. The optical fiber 124 extends in the proximal-to-distal direction and is inserted and axially displaceable within the fiber-receiving lumen 130 of the flexible needle device 118. The distal end portion 132 of the optical fiber is extendable out of the distal end portion 131 of the flexible needle device 118. In FIG. 1A and FIG. 1C, said end portion 132 is situated within the target tissue 108 (e.g., a cancerous lesion) and the needle-like distal tip 120 is shown to be exposed; this being for illustrative purposes, as in this state, further explained below, the distal tip 120 will be contained within the sheath 122 and only the distal end portion 132 is being exposed in the target tissue. As schematically illustrated in FIG. 1C, the distal end portion 132 of the optical fiber 124 has a light-diffusing section 136 configured for redirecting light that axially propagates in the optical fiber 124 in a proximal-to-distal direction from the light source 128 to be emitted to the surrounding target tissue 108. The diffuser section 136 may be configured for emitting light in a variety of directions such as non-directional scattered light, radial, spherical, radial along a section (e.g., along a predetermined irradiation length or portions thereof), radial along certain angular sectors (e.g., a 120-degree angular spread along its length), axial directions (e.g., front-face emission), and/or any clinically relevant combinations thereof. The light-diffusing section 136, in this embodiment, is flanked by two X-ray markers 138A and 138B, positioned at respective proximal and distal sides of the light diffuser 136. The X-ray markers 138A and 138B may, for example, be X-ray opaque annular or rod elements, e.g., made of gold or tungsten, disposed on the optical fiber 124. The X-ray markers serve for proper positioning of the optical fiber 124 within the target tissue 108 under real-time X-ray guidance imaging (e.g., immediately, or substantially immediately, upon imaging one or more said X-ray markers) and for verification using CT or cone-CT. In some cases, for effective treatment, the diffuser section 136 should, preferably, span the entire breadth of the target tissue 108, depending on the size of the desired ablation margin (in some instances, the PDT therapy may also able adjacent healthy tissue) or certainty to ablate the entire tumor. In other cases, the diffuser may extend beyond the tumor at least on one side or be completely contained within the tumor.

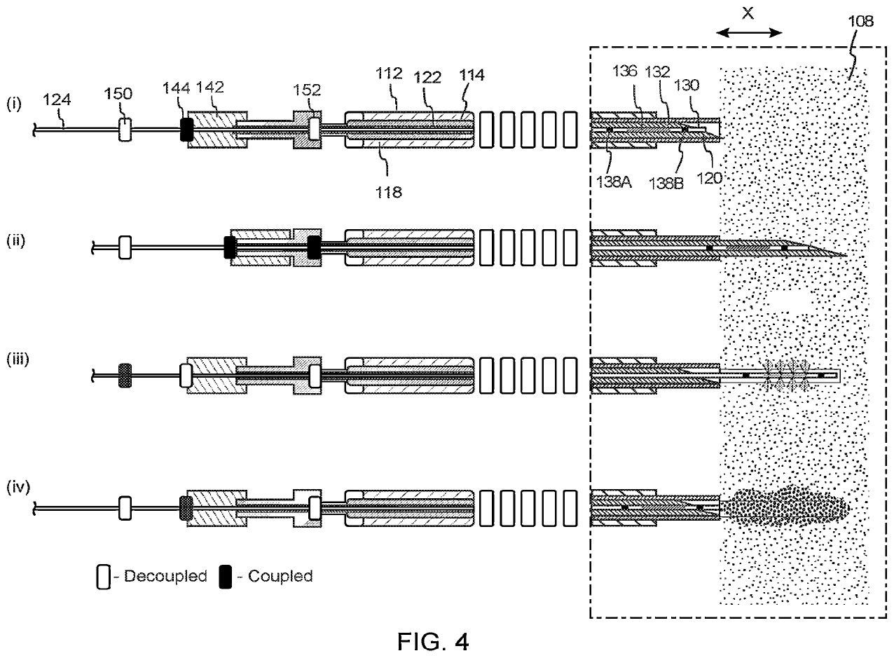

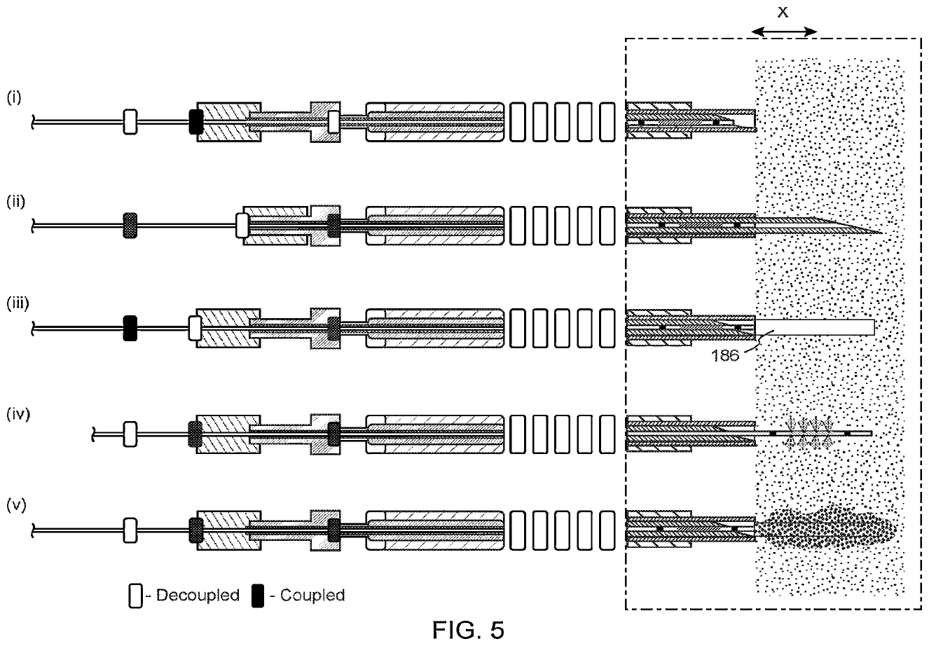

The displacement-control arrangement 102 is coupled to proximal portions of the delivery apparatus 104 including the flexible needle device 118, sheath 122, optical fiber 124 and delivery catheter 112, as can be seen in FIG. 2. The coupling mechanism, as will be further elucidated below, is configured to permit the joint axial displacement of some components of apparatus 104, for example, during initial deployment through the airways of the subject or through deployment of all components of the apparatus other than the catheter, through the catheter's working channel; and also to permit relative axial displacement of one component of apparatus 104 versus the other(s) to thereby advance through the different exemplary operational stages illustrated in FIGS. 4-5 and described in detail below.

Following deployment and upon arrival of a distal portion 106 of the delivery apparatus 104, particularly of the sheathed flexible needle device 114, to a site proximal to the target tissue 108, the displacement-control arrangement 102 is operated for axial forward displacement of the flexible needle device 114 and extracting the pointed, needle-like distal tip 120 for penetration of the target tissue 108. In some cases, the PDT system 100, can be configured for combination with a robotic system (e.g., for automatic or semi-automatic navigation and deployment of the delivery apparatus to the target tissue), and optionally for automatically performing one or more of the operational stages disclosed herein. In other cases, system 100 is configured for a partially combined operation with a robotic system, wherein some of the procedures, e.g., navigation and deployment, are performed automatically by the robotic system, and other procedures, e.g., one or more of the operational stages, described in detail below, are performed manually by a medical practitioner. In some cases, the catheter may be deployed first to the desired location utilizing a camera installed within the working channel. Once the distal end portion of the catheter is properly positioned, the camera may be removed from the working channel. Then, the sheathed flexible needle device containing the optical fiber is pushed through the catheter, until the distal tip of said sheathed flexible needle device is at least beyond the distal end portion of the catheter before retracting the sheath to expose the distal needle-like tip of the flexible needle device. In other cases, the fiber is a priori inserted into the lumen of the flexible needle device and deployed therewith through the catheter's working channel after the distal end portion of the catheter is positioned.

As can be seen in FIG. 2, the exemplary displacement-control arrangement 102 comprises a reciprocating block, generally designated 140, supported by a support frame 141, and comprises a handle 142, which is part of a flexible needle assembly that comprises the sheathed flexible needle device 114 and that can axially reciprocate, as represented by the bidirectional arrow 148, guided by track 149. The axial reciprocation of handle 142 causes a corresponding axial displacement of the sheathed flexible needle device 114 and can also cause, when the axial displacement of the sheath is arrested, e.g., by the tube coupling element 152 (which in this embodiment is a thumb screw), the relative axial displacement of the flexible needle device 118 vis-à-vis the sheath 122. The handle 142 is further connected to a fiber coupling element 144 that reciprocates therewith.

The displacement-control arrangement 102 also includes a fixed optical fiber engaging element 150 that is configured to arrest, when in the coupled state, the axial displacement of optical fiber 124 with respect to the delivery apparatus and/or the target tissue. By coupling/decoupling of elements 144, 152, and 150 the reciprocal movement of the handle 142 can be translated into relative axial displacement of the optical fiber 124 and flexible needle device 118, respectively, as will be further described below. It is to be noted, according to an embodiment of this disclosure, that the coupling of the tube coupling element 152 to a proximal portion of the flexible needle device 118 and coupling of the optical fiber coupling element 144 to the optical fiber 124 are independent of one another permitting discrete axial displacement of the sheathed flexible needle device 114 (or only the flexible needle device without the sheath) and the optical fiber 124.

A catheter-coupling element 154 is configured to fix the proximal end of delivery catheter 112 to displacement-control arrangement 102. The displacement control arrangement 102 is integrated, through connecting member 155 of frame 141, with a disposable robotic-interface element 156 of a robotic-assisted bronchoscope system such as, for example, the ION Endoluminal System (e.g., Model IF1000) by Intuitive Surgical Inc., USA. The robotic system is configured to guide delivery catheter 112, being fixed, once in position, by means of the coupling element 154.

Displacement limiting element 146 is configured to limit the displacement of handle 142, depending on its axial position, for example, to permit an axial displacement of 1 cm, 2 cm, 3 cm, etc.

In accordance with the above specific embodiment, for example, upon engagement of element 150 with optical fiber 124, while element 144 is decoupled from optical fiber 124, and element 152 is coupled to flexible needle device 118, the forward displacement of handle 142 will result in the forward axial displacement of flexible needle device 118 without axial displacement of optical fiber 124. In contrast, when element 150 is disengaged from optical fiber 124 and, while element 144 is coupled to optical fiber 124, the forward movement of handle 142 will cause the forward axial displacement of optical fiber 124 while displacing flexible needle device 118. The opposite occurs in the case of a rearward displacement. In this embodiment, coupling element 144 is a magnetic flapper type clamp but can also, by other embodiments, be a Touhy Borst adapter fitted to the proximal end of flexible needle device 118 that would, in this case, axially extend through displacement-control arrangement 102 with its proximal end being projected rearward from the handle 142.

Another embodiment of a system of this disclosure is depicted in FIGS. 3A-3B. Like or analogous elements that serve a similar function to corresponding ones in FIG. 1A-2 have been marked by the same reference numerals shifted by 100 (for example, optical fiber 224 is similar in function to optical fiber 124 of FIGS. 1A-2, tube coupling element 252 serves an analogous function to that of the tube coupling element 152 of FIGS. 1A-2, etc.). To better understand the functionality of elements in FIGS. 3A-3B, the reader may refer to the description of FIGS. 1A-2; and vice versa. One distinct feature of the embodiment of FIG. 3A-3B is that the entire flexible needle assembly can be pushed beyond the distal end portion of the delivery catheter, while allowing for extraction of the flexible needle assembly with the optical fiber remaining fixed in position.

In this embodiment, use is made of a delivery apparatus 204 that comprises a delivery catheter 212, a sheathed flexible needle device 214, of which the tubular sheath 222 can be seen, and a manipulation element 260, which is integral with the sheathed flexible needle device, and wherein the sheathed flexible needle device 214 and manipulation element 260, taken together, constitute a flexible needle device assembly. An example of such an assembly is FleXeneedle (e.g., Model 10005) by Broncus Medical, Inc., USA. The displacement-control arrangement 202, includes a reciprocating block 240, that can axially reciprocate on a track 249, the axial reciprocation being represented by bidirectional arrow 248.

Manipulation element 260 is fixed to frame 241 by means of a coupling member 262. The sheathed flexible needle device of which sheath 222 is seen, loops backwards through the displaceable tube coupling element 274 (that is part of reciprocating block 240), displacement limiting element 246 (serving to limit the displacement distance of block 240), fixed tube coupling element 252, and into catheter 212, fixed to frame 241 by means of catheter coupling element 254. Fitted at the proximal end of manipulation element 260, is an optical fiber engaging element 270, which in this specific embodiment is a Touhy Borst adapter. When adapter 270 is closed, this being achieved through screw-tightening, the optical fiber 224 is in a fixed axial position with respect to the sheathed flexible needle device and cannot be axially displaced with respect thereto. If opened, upon displacement of block 240 in a rearward direction (namely, away from the disposable robotic-interface element 256) the optical fiber 224 can be axially displaced with respect to the sheathed flexible needle device.

The relative axial displacement of the flexible needle device (and its distal needle-like tip) with respect to the sheath 222 can be achieved by manipulation element 260 through displacing its handle 264 in a rearward direction as represented by arrow 280. If adaptor 270 is in its closed state, such axial displacement of the flexible needle device will be accompanied by joint axial displacement of the optical fiber 224.

Engagement of the displaceable tube coupling element 274 with sheath 222 permits axial displacement of sheath 222 vis-à-vis other axially displaceable elements of delivery apparatus 204, in line with the reciprocal axial displacement of block 240. The proximal end of sheath 222, with its terminal portion 272, is fixed to coupling member 262. Accordingly, the axial displacement of sheath 222 will also cause a corresponding axial displacement of the flexible needle device that is fixed to handle 260, which is, in turn, fixed to coupling member 262.

The fixed tube coupling element 252 is configured to fix the sheath vis-à-vis the catheter 212 and the disposable robotic-interface element 256. Once it engages sheath 222, axial displacement of the optical fiber 224 through engagement of fiber coupling element 244, with both adapter 270 and displaceable tube coupling element 274 being open, the axial displacement of block 240 will cause corresponding axial displacement of the optical fiber 224 with respect to the flexible needle device and the catheter 212.

Through engagement/disengagement of elements 244, 252, 274, and adapter 270, in any clinically relevant combination thereof, as well as manipulation of manipulation element 260, the relative axial displacement of the various axially displaceable components of delivery apparatus 204 one versus the other and in accordance with the relevant operational stage, some of which are elaborated on below, is achieved.

Schematically depicted in FIGS. 4-5, are four and five operational stages, respectively, of two treatment PDT procedure modes according to embodiments of this disclosure. Further depicted are the corresponding mechanistic outcomes of these operational stages within the target tissue. For ease of description, use is made of the same reference numerals used in FIGS. 1A-2 which, for visual clarity are shown only in FIG. 4—operational stage (i) although they refer to the same signified components as they appear elsewhere throughout FIGS. 4-5.

Turning to FIG. 4, operational stage (i) describes a deployment stage, wherein the sheathed flexible needle device 114, accommodated within the working channel 118 of a delivery catheter 112, is deployed, jointly with the optical fiber 124 such that its distal end comes into proximity of the target tissue 108. Hence the fiber coupling element 144 is in the coupled state, and the fixed optical fiber engaging element 150 as well as tube coupling element 152 is in a decoupled state and the described components are advanced together along the delivery axis X. Upon the distal end of the sheathed flexible needle device 114 reaching the target tissue, the tube coupling element 152 is switched into the coupled state, and operational stage (ii) which is a tissue penetration stage, is carried out in which the flexible needle device 118 and the optical fiber 124 are axially forwardly displaced, out of sheath 122, to an extended state, to permit the distal tip 120 to penetrate the tissue. Then, at a next operational stage (iii), being an exposure stage, the fiber coupling element 144 is switched to the decoupled state, element 150 is switched to the coupled state and element 152 is switched to the decoupled state, and by pulling handle 142 backwards, the tip 120 of the flexible needle device 118 is retracted in a proximal direction back into sheath 122 to thereby expose the distal end portion 132 of the optical fiber 124 comprising the light-diffusing section 136 in the tissue and to retain it therein. Then, in stage (iv), being an internalization stage, following irradiation at the target tissue, by switching element 150 into the decoupled state and element 144 into the coupled state, and pulling handle 142 backwards, the diffuser section 136 is pulled back into the fiber-receiving lumen 130 of the flexible needle device 118. The entire delivery apparatus 104 can then be retracted.

Attention is now drawn to FIG. 5, wherein operational stage (i) is a deployment stage, wherein the sheathed flexible needle device 114, accommodated within the working channel 118 of a delivery catheter 112, is deployed with the optical fiber 124 contained within, until the distal end of the sheathed flexible needle device 114 comes into proximity of the target tissue 108. During such deployment, tube coupling element 152 is in the decoupled state, fiber coupling element 144 is in the coupled state and element 150 is in the decoupled state, enabling the sheathed flexible needle device 114 with the optical fiber 124 to advance along the delivery axis X in a proximal-to-distal direction. Upon positioning of the sheathed flexible needle device 114 proximate to the target tissue 108, four consecutive operational stages may follow, wherein: (ii) depicts a tissue penetration stage in which the flexible needle device 118 is axially forwardly displaced out of sheath 122, to the forward state to cause the distal tip 120 to penetrate the target tissue; (iii) depicts a retraction stage in which the flexible needle device 118 is axially rearwardly displaced to retract the distal tip 120 from the tissue and back into sheath 122; (iv) depicts a fiber placement stage in which the optical fiber 124 is axially forwardly displaced into a tissue penetrating channel 186 formed by the distal tip 120, and retained therein, and; (v) depicts an internalization stage for retracting the diffuser section 136 back into the fiber-receiving lumen 130 of the flexible needle device 118 following irradiation at the target tissue.

As can be further appreciated in FIG. 5, in the tissue penetration stage (ii) and in retraction stage (iii) elements 150 and 152 are in a coupled state and element 144 in a decoupled state, whereupon forward displacement of handle 142 axially displaces the flexible needle device 118 to cause the needle-like tip 120 to penetrate the tissue and the consequent rearward displacement causes the needle-like tip 120 to retract back into the sheath 122. In the fiber placement stage (iv) and in the internalization stage (v), tube coupling element 152 and optical fiber coupling element 144 are in their coupled state, while element 150 is in the decoupled state, whereupon forward axial displacement of the handle 142 causes distal portion 132 of the optical fiber 124 to be extracted into the tissue penetrating channel 186 and subsequently internalized upon counter displacement of the handle.

It should be noted, that regardless of the chosen deployment sequence to be performed, the diffuser section 136, as described in FIGS. 4-5, and according to an embodiment of this disclosure, may comprise two flanking X-ray markers 139A and 139B, for correct and safe positioning of the diffuser section 136. Options for placement of these markers include within the target tissue, at least one outside or both outside of said target tissue.

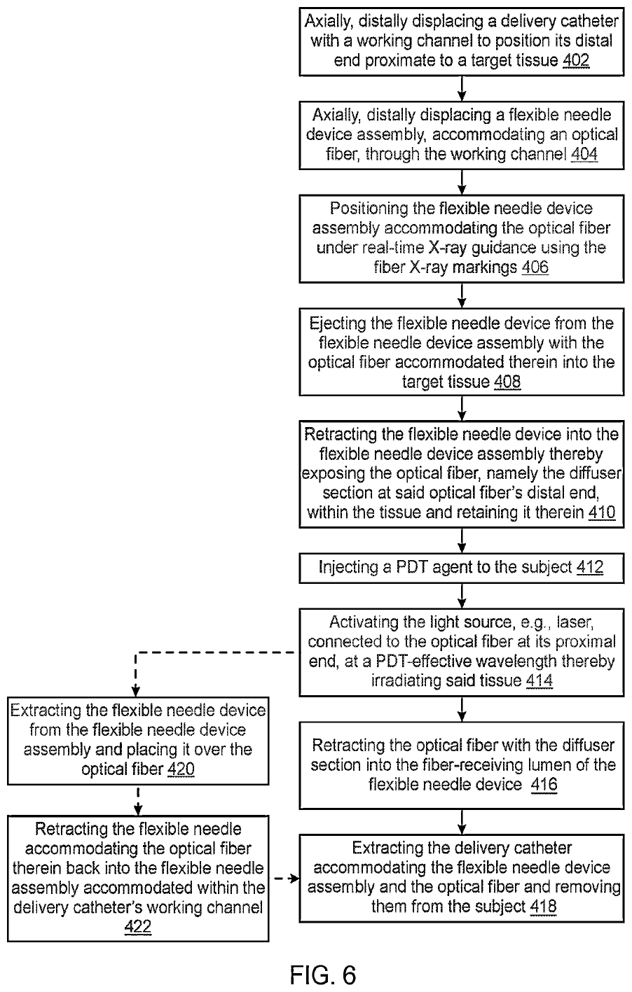

Turning now to FIG. 6, depicted is a general flowchart of a method 400 for PDT of a target tissue of a subject comprising different method elements, as will be shortly described, that may be carried out in any clinically applicable order other than that described herein, which pertains to but one embodiment of the present disclosure. For example, method element 412, wherein the PDT agent is administered to the subject, may occur either before, during, or after such deployment. Furthermore, as noted above, the different components of the PDT assembly, can be assembled a priori to deployment within the subject, or in situ, in varying combinations.

The PDT method 400 particularly describes, by way of non-limiting example, the use of a delivery catheter for the deployment of a sheathed flexible needle device, wherein the former is deployed first to a site proximate to the target tissue, and the a priori assembled sheathed flexible needle device is deployed within the delivery catheter's working channel thereafter.

A PDT method 400, is performed by axially displacing in a distal direction, a delivery catheter with a working channel to position its distal end proximate to a target tissue, as described in method element 402. Then, in method element 404, a sheathed flexible needle device having a pointed, needle-like distal tip and a fiber-receiving lumen, accommodating therein an optical fiber, is extended through the delivery catheter's working channel in a proximal-distal direction, along a delivery axis. The sheathed flexible needle device, having the optical fiber accommodated therein, is then positioned at the target tissue under real-time X-ray guidance, as described by method element 406, using the X-ray markers flanking the diffuser section at the distal end portion of the optical fiber. In the following method element 408, the flexible needle device, accommodating the optical fiber, is ejected from the sheathed flexible needle device, particularly from the tubular sheath component, in a distal direction to penetrate the target tissue with its pointed, needle-like distal tip. The flexible needle device is then retracted along the delivery axis in a proximal direction, from the tissue back into the sheath of the sheathed flexible needle device, in method element 410, to thereby expose the diffuser section of the optical fiber and retain it within the tissue. According to this specific embodiment, the PDT agent, typically, albeit not exclusively, Padeliporfin (WST11) is then administered to the subject, typically intravenously, in method element 412. Following the administration of the PDT agent, the light source, typically a laser, configured for emitting light a PDT-effective wavelength, applicable for said PDT method 400, is then activated, as described in method element 414. The light source is connected to the proximal end of the optical fiber, wherein, upon its activation, light propagates through the optical fiber in an axial direction and upon reaching the diffuser section positioned within the target tissue is, typically, albeit not exclusively, scattered in a mostly radial direction to irradiate the surrounding target tissue. Upon completion of the desired duration of light irradiation, the light source is deactivated and the optical fiber is retracted back in a proximal direction into the sheathed flexible needle device to remove the light diffuser section from the irradiated tissue, as described in method element 416. Once the optical fiber is nestled safely within the sheathed flexible needle device, the delivery catheter accommodating the sheathed flexible needle device is withdrawn to remove the PDT assembly from the subject in method element 418.

PDT method 400, may, by another embodiment, comprise other method elements, for example, method elements 420 and 422, wherein, following the irradiation step provided by method element 414: the flexible needle device is extended in a distal direction once again out of the sheath of the sheathed flexible needle device and placed over the optical fiber, still retained within the target tissue; and then the flexible needle device, now accommodating the distal end portion of the optical fiber, is retracted in a proximal direction back into the sheath of the sheathed flexible needle device. These exemplary method elements may, optionally, be incorporated into a PDT method to ensure an additional layer of safety.

1. A PDT assembly for use in photodynamic therapy (PDT) of a target tissue, comprising delivery apparatus:
a delivery apparatus that comprises:
(i) a flexible needle device comprising an elongated flexible tube having a fiber-receiving lumen, extendable in a proximal-distal direction along a delivery axis, and a pointed, needle-like distal tip for penetrating said tissue; and
(ii) an optical fiber insertable and axially displaceable within said lumen and couplable at its proximal end to a light source, emitting a PDT-effective light at a wavelength applicable for said PDT, to permit light from said source to enter the optical fiber, and said fiber having a distal end portion with a light-diffusing section for emitting light from within the fiber in one or more directions, said diffuser having one or more X-ray markers; and comprising
a displacement-control arrangement couplable to a proximal portion of the flexible needle device and to a proximal portion of the optical fiber accommodated within the flexible needle device with the proximal portion projecting out of the proximal end of the flexible needle device, and configured for selective axial displacement of said flexible needle device and said optical fiber one versus the other.

2. (canceled)

3. The PDT assembly of claim 1, comprising two X-ray markers flanking the light-diffusing section.

4. The PDT assembly of claim 1, wherein said diffuser section is configured for emitting light in one or both of a radial and axial directions.

5. (canceled)

6. (canceled)

7. The PDT assembly of claim 1, wherein at least one of the X-ray markers is an X-ray opaque annular element.

8. The PDT assembly of claim 1, comprising an X-ray opaque rod element at a distal end portion of the optical fiber.

9. The PDT assembly of claim 1, wherein a distal portion of said flexible needle device is highly flexible, such that it can bend, without loss of its integrity, to define a curvature with a radius of 20 mm or less.

10. The PDT assembly of claim 1, wherein the displacement-control arrangement is configured for:
(i) retaining said distal tip within the delivery apparatus during deployment to a site proximal to said tissue; and
(ii) axially displacing the flexible needle device and extracting said distal tip following deployment.

11. The PDT assembly of claim 1, wherein the displacement-control arrangement comprises a tube coupling element that can axially reciprocate with respect to other said displacement-control arrangement elements, between a rearward state and a forward state, and having a coupled state in which it is physically coupled to a proximal portion of said flexible needle device for its respective axial displacement in both proximal and distal directions along said delivery axis, and having a decoupled state in which the axial displacement of said tube coupling element does not cause displacement of said flexible needle device.

12. The PDT assembly of claim 1, wherein the displacement-control arrangement comprises an optical fiber coupling element that can axially reciprocate, with respect to other said control device elements, between a retracted state and an extended state, and having a coupled state in which it is physically coupled to said optical fiber for respective axial displacement of the fiber in both proximal and distal directions along said delivery axis, and having a decoupled state in which the axial displacement of said optical fiber coupling element does not cause displacement of said optical fiber.

13. The PDT assembly of claim 12, wherein:
the displacement-control arrangement comprises a tube coupling element that can axially reciprocate, with respect to other said control device elements, between a rearward state and a forward state, having a coupled state in which it is physically coupled to a proximal portion of said flexible needle device for its respective axial displacement in both proximal and distal directions along said delivery axis, and having a decoupled state in which the axial displacement of the tube coupling element does not cause displacement of said flexible needle device; and wherein
the coupling of the tube coupling element to a proximal portion of said flexible needle device and coupling of the optical fiber coupling element to said optical fiber are independent of one another permitting discrete axial displacement of the flexible needle device and the optical fiber.

14. The PDT assembly of claim 13, wherein said tube coupling element and said optical fiber coupling element are physically coupled to one another to define a reciprocating block.

15. (canceled)

16. The PDT assembly of claim 15, wherein the displacement-control arrangement has operational modes that comprise:
(i) a tissue penetration mode in which the flexible needle device and the optical fiber are axially displaced in a distal direction to an extended state to permit said distal tip to penetrate said tissue;
(ii) an exposure mode in which said flexible needle device is retracted in a proximal direction to expose said optical fiber's distal end portion in said tissue and retain it therein; and
(iii) an internalization mode for retracting said end portion to within said flexible needle device.

17. The PDT assembly of claim 16, wherein:
in said tissue penetration mode, the tube coupling element and the optical fiber coupling element are both in their coupled state for joint axial displacement of the flexible needle device and the optical fiber in a distal direction along said delivery axis to reach the extended state for penetration of the target tissue; and wherein
in said exposure mode, the tube coupling element is in the coupled state and the optical fiber coupling element is in the decoupled state for retracting said flexible needle device's pointed distal tip, to thereby expose said optical fiber's distal end portion.

18. The PDT assembly of claim 15, wherein the displacement-control arrangement has operational modes that comprise:
(i) a tissue penetration mode in which the flexible needle device is axially displaced in a distal direction to the forward state to cause its pointed, needle-like distal tip to penetrate the target tissue;
(ii) a retraction mode in which said flexible needle device is axially displaced in a proximal direction to retract said distal tip from said tissue;
(iii) a fiber placement mode in which said optical fiber is axially displaced in a distal direction into a channel formed in said tissue by said distal tip; and
(iv) an internalization mode for retracting said end portion to within said flexible needle device.

19. The PDT assembly of claim 18, wherein:
in said tissue penetration mode and said retraction mode the tube coupling element is in its coupled state for axial displacement of said distal tip in a distal direction to the forward state for penetrating the target tissue, and in the opposite direction, respectively; and wherein
in said fiber placement mode the optical fiber coupling element is in its coupled state for axial displacement of the optical fiber in a distal direction to the extended state for positioning said distal end portion of the optical fiber within said tissue.

20. A system for photodynamic therapy (PDT) in a target tissue, comprising an assembly of claim 1, and a light source emitting light at a wavelength applicable for said PDT and connectable to said optical fiber.

21. (canceled)

22. A method for deploying a light-diffusing section of an optical fiber into a subject's target tissue for photodynamic therapy (PDT), comprising:
axially displacing in a distal direction, a flexible needle device extending in a proximal-distal direction along a delivery axis, said device having an elongated tube with a pointed, needle-like distal tip and having a fiber-receiving lumen to position said distal tip at a site proximal to said tissue;
axially displacing in a distal direction, said flexible needle device to pierce and penetrate said tissue;
axially displacing said flexible needle device in a proximal direction to retract said distal tip from said tissue thereby defining a tissue penetrating channel in the tissue; and
axially displacing in a distal direction, through said fiber-receiving lumen, an optical fiber to position the distal end portion of said optical fiber in said penetration channel; said optical fiber connected at its proximal end to a light source that emits PDT-effective light to permit light from said light source to enter said optical fiber, and further having a light-diffusing section at its distal end portion for emitting said light in one or more directions.

23. The method of claim 22, wherein the axial displacement of said flexible needle device is within a lumen of a delivery catheter.

24. The method of claim 23, wherein the distal end portion of said optical fiber comprises one or more X-ray markers, and wherein the axial displacement of said flexible needle is performed under real-time X-ray imaging guidance.

25. A method for photodynamic therapy (PDT) of a target tissue of a subject, comprising:
axially displacing in a distal direction, a delivery catheter with a working channel that extends in a proximal-distal direction and defines a delivery axis to position the distal end of the delivery catheter proximal to said tissue;
axially displacing in a distal direction, through the working channel, a flexible needle device having a pointed, needle-like distal tip and a fiber-receiving lumen accommodating therein an optical fiber, to cause said tip to pierce the target tissue and lead the distal end of the flexible needle device with the distal end portion of the optical fiber to penetrate said tissue; said optical fiber being connected at its proximal end to a light source that emits PDT-effective light to permit light from said light source to enter said optical fiber, and further having a light-diffusing section at its distal end portion for emitting said light in one or more directions;
axially displacing said flexible needle device in a proximal direction to retract distal said distal tip and to expose said diffuser section and retain it within said tissue;
injecting a PDT agent to the subject;
activating said light source to thereby irradiate said tissue with said PDT-effective light emitted from said diffuser section; and
removing said delivery catheter with said flexible needle device and said optical fiber from said subject.

26. (canceled)
